(19) United States
(12) Patent Application Publication
(10) Pub. No.: US 2004/0225245 A1
(43) Pub. Date: Nov. 11, 2004

(54) Knee brace with directional elastic

(76) Inventor: Ronald E. Nelson, Cambridge, MN (US)

Correspondence Address: FOLEY & LARDNER, 150 EAST GILMAN STREET, P.O. BOX 1497, MADISON, WI, 53701-1497, US

(21) Appl. No.: 10/429,951

(22) Filed: May 5, 2003

(51) Int. Cl. A61F 5/00

(52) U.S. Cl. 602/26

(57) ABSTRACT

A knee brace for use by athletes or others requiring protection and support of the knee. A base is configured to closely fit around front, side, and back portions of the knee joint and adjacent upper and lower leg portions. The portions of the base located adjacent to the front portions of the knee joint are made of elastic panels that stretch primarily in the vertical direction, so that the base conforms closely throughout the entire range of motion of the knee joint. The portions of the base located adjacent to the side and back portions of the knee joint can be made of elastic panels that stretch primarily in the horizontal direction. Upper and lower support strap segments and upper and lower fastening straps may also be provided.

### FIELD OF THE INVENTION

This invention relates generally to the field of articles worn by persons to reduce the likelihood, severity, or exacerbation of injury to the body, and more specifically to the field of braces worn on the knee.

### BACKGROUND OF THE INVENTION

Flexible knee braces are used by athletes and other persons engaged in vigorous physical activity to protect the knee from injury and to avoid exacerbation of existing injury. The knee is one of the most heavily used joints of the body, as it is used in any activity that involves walking or running. The knee is also a common subject of injury, due to the relatively high levels of stress it must bear under dynamic loads that are often multiples of the entire weight of the body. During normal ambulation, in occupations involving physical labor, and especially during strenuous sports, the knee can undergo abnormal motions as a result of quick changes in direction, fatigue, uneven surfaces, or impacts. These abnormal motions can cause sprains or more serious injuries, such as dislocation, stretching, or tearing of the tissues that make up the knee.

For these reasons, devices to protect the knee against abnormal motions have been used for many years, in a variety of specific embodiments which vary in their abilities to protect against different types of abnormal motions. Such devices may also provide additional benefits such as insulating the knee to keep it warm, protecting the knee against impact, or compressing the knee to reduce discomfort.

The protections afforded by such devices are often accompanied by reductions in range or ease of normal motion. These devices can also have other undesirable aspects such as added weight on the leg, potential for self-injury or injury to others caused by rigid components, difficulty of application and removal, cost, appearance, irritation or chafing of the skin, and other drawbacks.

Some prior art devices utilize a sleeve structure, and this approach can have a number of benefits and advantages. Such sleeve-based devices may be relatively comfortable, and they can be inexpensive to manufacture. Sleeve-based devices may provide compression, and they can also help to keep the knee joint warm.

Such sleeve-based devices can be implemented as a tubular sleeve of elastic material, or as a reclosable sleeve that can be fastened about the knee area. Devices implemented as a tubular sleeve must typically be manufactured in a range of sizes to accommodate knees of varying dimensions, which can increase manufacturing and distribution costs, as well as the cost of shelf space. Reclosable sleeves can often be manufactured in one or two sizes and still fit the majority of people, so the reclosable sleeve approach is often preferred.

Although sleeve-based devices have a number of advantages, by themselves such devices may provide only limited support and protection against abnormal motions. For this reason, some sleeve-based devices include semi-rigid stays, which are flexible in some directions but relatively rigid in other direction. Such stays can provide support against abnormal motions beyond that available from the sleeve by itself.

Prior sleeve-based devices have been designed so that all the elastic pieces stretch in the horizontal direction. Because the vertical and horizontal dimensions of the knee change as the knee moves through its ordinary flexion and extension movements, in order to maintain a good fit the vertical and horizontal dimensions of a sleeve-based knee brace must also change. However, in these prior sleeve-based devices the elastic pieces in the area at the front of the knee cannot stretch vertically in order to conform properly, so the closeness of fit of such a knee brace deteriorates as the knee moves through its ordinary range of motion. The lack of vertical stretch in the elastic pieces in the area at the front of the knee also causes these devices to resist the normal movement of the knee, thereby reducing athletic performance and mobility.

Other prior sleeve-based devices, whether based on tubular elastic sleeves or reclosable elastic sleeves, have been designed using four way stretch elastic, so that all the elastic pieces stretch in both horizontal and vertical directions. Because all the elastic in these prior sleeve-based devices stretches in all directions, these devices provide relatively limited protection against abnormal movement.

### SUMMARY OF THE INVENTION

A preferred embodiment of a knee brace according to the present invention includes elastic panels at the front of the knee which stretch primarily in the vertical direction, and elastic panels at the sides and rear of the knee which stretch primarily in the horizontal direction. Such a knee brace can conform well throughout the normal range of motion of the knee, to improve support and protection and reduce any negative impact on athletic performance caused by wearing the brace.

The present invention includes a base which can be worn in snug covering relationship to the knee and adjacent portions of the leg of a person. This base can be an openable sleeve which can be fastened about the knee and adjacent portions of the leg, but it can also be a tube that can be slipped onto the knee and adjacent portions of the leg. The base can include an opening at the rear to avoid bunching or undue restriction of movement, but this is not required. All such alternative embodiments will be referred to herein as a base.

Whether the base is formed as an openable sleeve or as a tube, the base of a knee brace according to the invention includes one or more panels, located at the front of the knee when the brace is worn, that are made of elastic which stretches primarily in the vertical dimension. The base can also include one or more panels, located at the sides or rear of the knee when the brace is worn, made of elastic that stretches primarily in the horizontal direction.

In a preferred embodiment, the base is a reclosable sleeve having a central panel made of elastic that stretches primarily in the horizontal direction and two side panels made of elastic that stretches primarily in the vertical direction. When the brace is worn, the central panel forms the rear and sides of the brace, and the side panels form the front of the brace. Hook and loop fastener material is preferably used to fasten the base about the knee, such that the tightness and position of the base can be adjusted without removing it entirely. This also allows the base to be manufactured in a single size which can be adjusted to fit a wide range of people.

One or more fastening straps can be used to secure the base to portions of the leg. Preferably, these fastening straps include inelastic sections at the front and sides of the leg, and elastic sections at the rear of the leg which stretch primarily in the horizontal direction.

One or more upright support members can be attached to the sides of the base, to provide additional support and protection against abnormal motions. Preferably, these upright support members may be semi-rigid stays made of compressed spring structures, but they may also be semi-rigid stays made of resilient plastic or similar material. The upright support elements may also be rigid hinges, or they may be a combination of semi-rigid stays and hinges.

The knee brace of the invention preferably incorporates a circular opening for the patella and crossed support straps or support strap segments to provide direct patella stabilization. In addition to providing direct patella stabilization, the circular opening can help to locate the brace properly relative to the patella.

Further objects, features, and advantages of the invention will be apparent from the following detailed description when taken in conjunction with the accompanying drawings.

### BRIEF DESCRIPTION OF THE DRAWINGS

In the drawings:

FIG. 1 is a front view of a knee brace according to the invention fitted on the leg of a person;

FIG. 2 is a side view of a preferred embodiment of a knee brace according to the invention fitted on the leg of a person;

FIG. 3 is a side view of an alternate embodiment of a knee brace according to the invention fitted on the leg of a person;

FIG. 4 is an outside view of a knee brace according to the invention laid flat;

FIG. 5 is an inside view of a knee brace according to the invention laid flat;

FIG. 6 is an inside exploded view of a knee brace according to the invention laid flat;

FIG. 7 is a front view of an exemplary resilient stay member;

FIG. 8 is a side view of an exemplary resilient stay member;

FIG. 9 is a side view of an exemplary hinge;

FIG. 10 is a front view of an exemplary hinge; and

FIG. 11 is a cross-sectional view of the knee brace of FIG. 1 taken along the line 11-11 thereof.

### DETAILED DESCRIPTION OF THE INVENTION

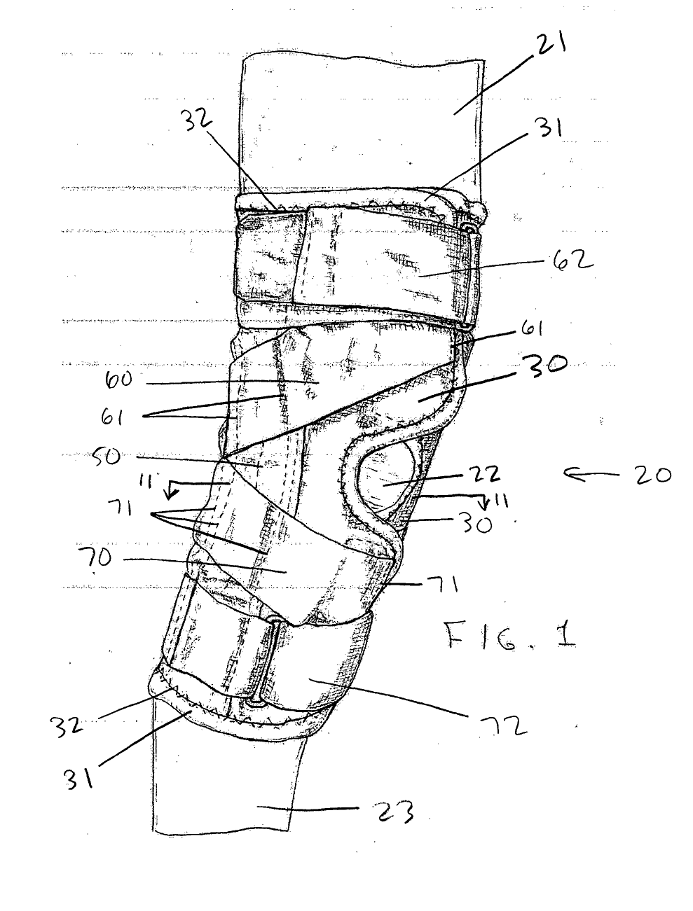

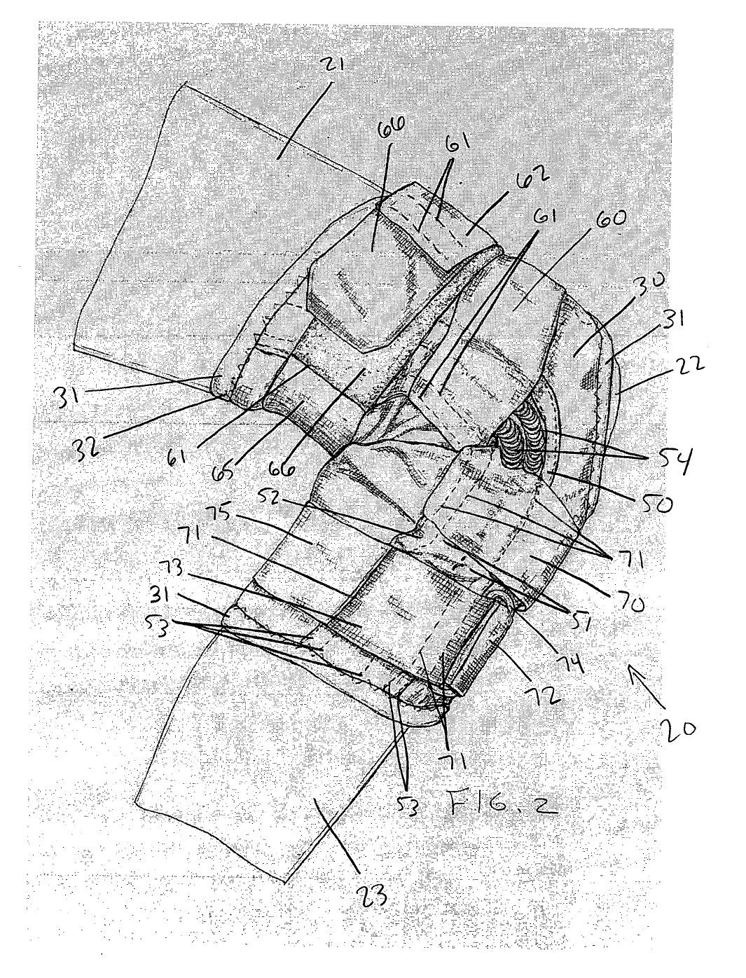

Referring to the drawings, FIGS. 1 and 2 show a preferred embodiment of a knee brace according to the invention indicated generally at 20 worn on the leg of a person. The knee brace 20 includes a base 30 which preferably forms a reclosable sleeve that can be fastened about the knee, including portions of the upper leg 21, the kneecap 22, and portions of the lower leg 23.

As best shown in FIG. 6, the base 30 of the knee brace 20 includes a center panel 80 formed of elastic that stretches primarily in the horizontal direction, and two side panels 81 formed of elastic that stretches primarily in the vertical direction. When the knee brace 20 is worn by a person, the two side panels 81 include a front portion that is positioned across the front of the leg, so that the elastic forming the side panels 81 stretches primarily in a vertical direction from the upper leg of the person to the lower leg of the person. When the knee brace 20 is worn by a person, the center panel 80 includes a rear portion that is positioned across the rear of the leg, so that the elastic forming the center panel 80 stretches primarily in a horizontal direction around the leg.

The knee brace may include one or more upright support assemblies, indicated generally at 50. The knee brace may include one or more upper support strap segments, indicated generally at 60, and lower support strap segments, indicated generally at 70. The knee brace may also include one or more upper fastening straps, indicated generally at 62, and lower fastening straps, indicated generally at 72.

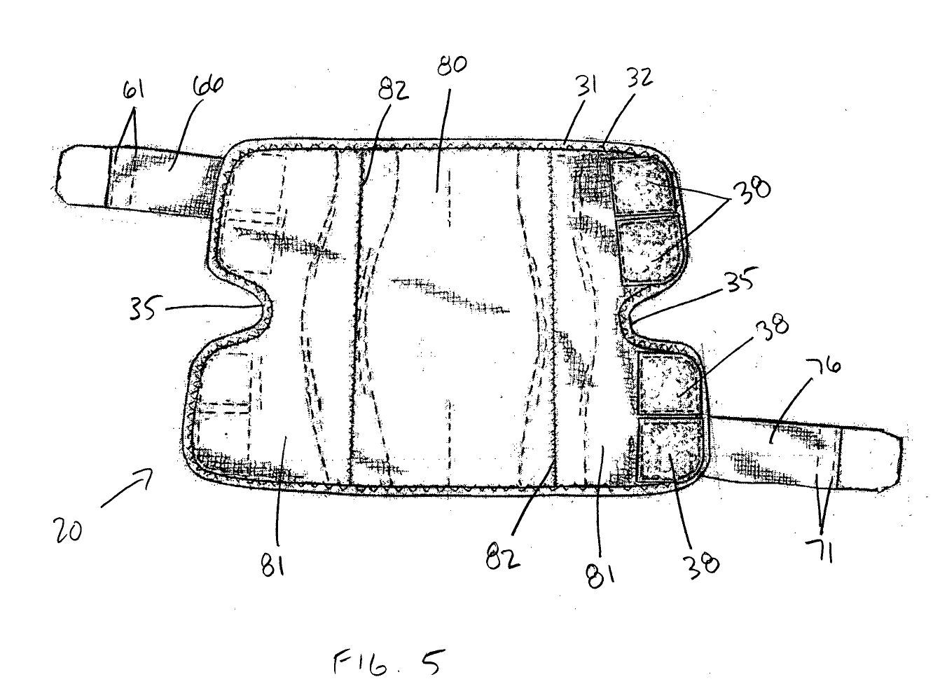

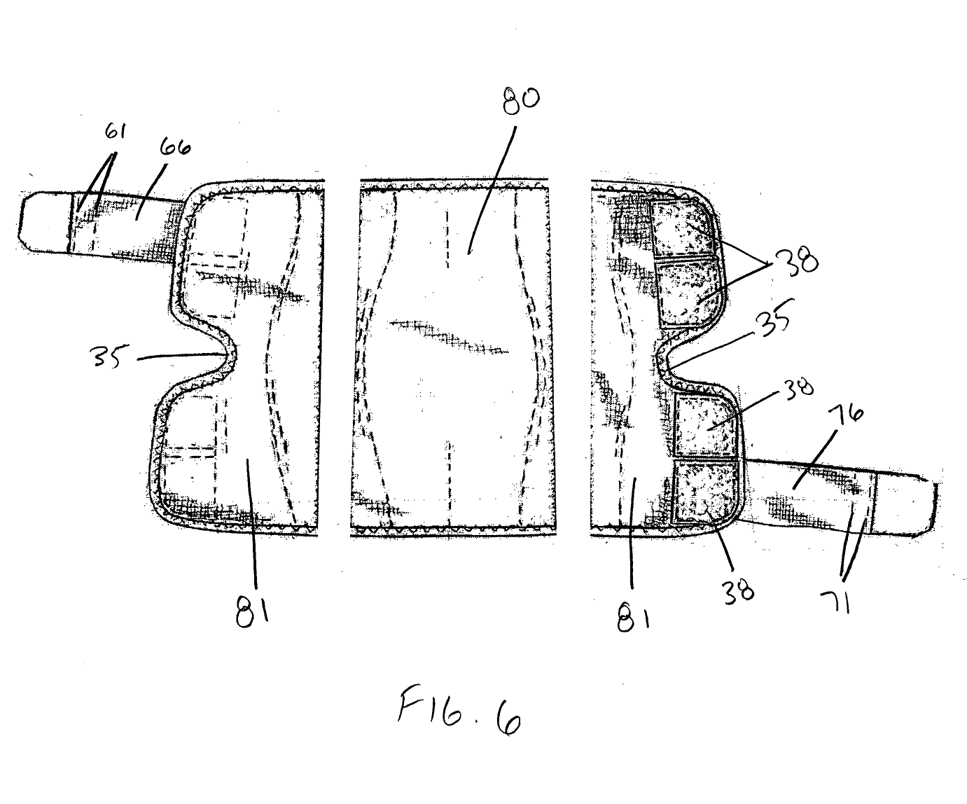

As best shown in FIGS. 2 and 3, one or more upright support assemblies 50 can be attached to the base 30. As shown in the preferred embodiment of FIG. 2, an upright support assembly 50 can be formed using one or more elongated pockets 51 containing an upright support member, for example a resilient stay 54. The resilient stay 54 can be made of a flattened spiral core of stainless steel, as best shown in FIGS. 7 and 8.

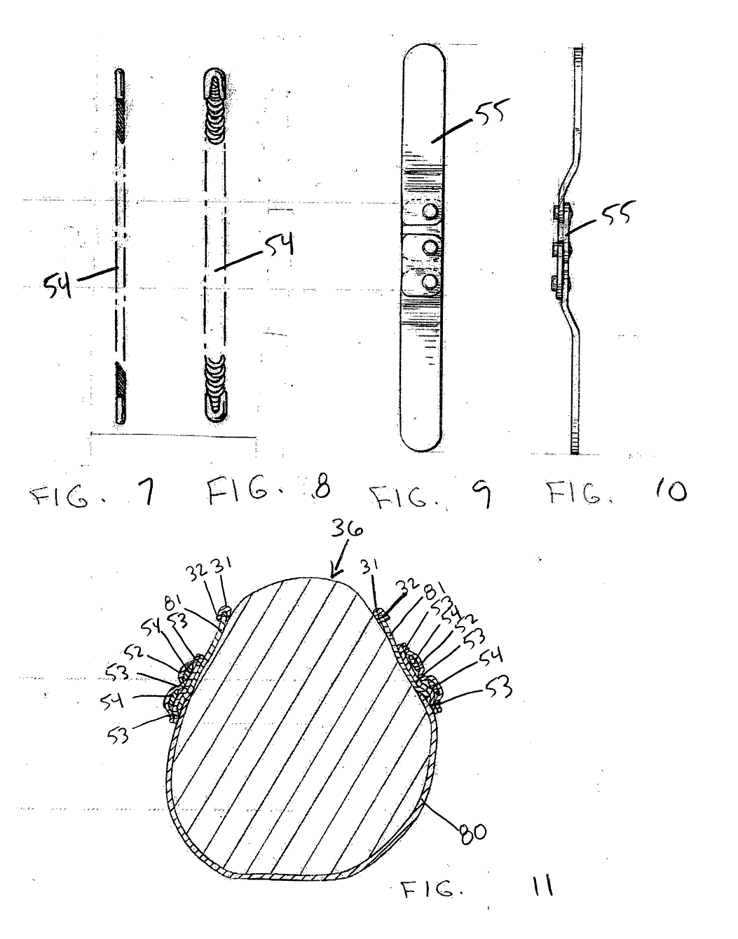

As shown in the alternative embodiment of FIG. 3, an upright support assembly 50 can also be formed using one or more elongated pockets 51 containing a hinge 55. The hinge 55 can be, for example, a hinge of the types shown in U.S. Pat. No. 4,726,362 to Nelson, or U.S. Pat. Nos. 4,573,455 or 4,844,057 to Hoy, as best shown in FIGS. 9 and 10.

An elongated pocket 51 of an upright support assembly 50 can be formed between stitches 53 that fix a pocket cover strip 52 to the base 30. The pocket cover strip 52 is preferably made of the same elastic sheet material (that stretches in the horizontal direction only) as the center panel 80 that forms the rear of the base 30 when worn.

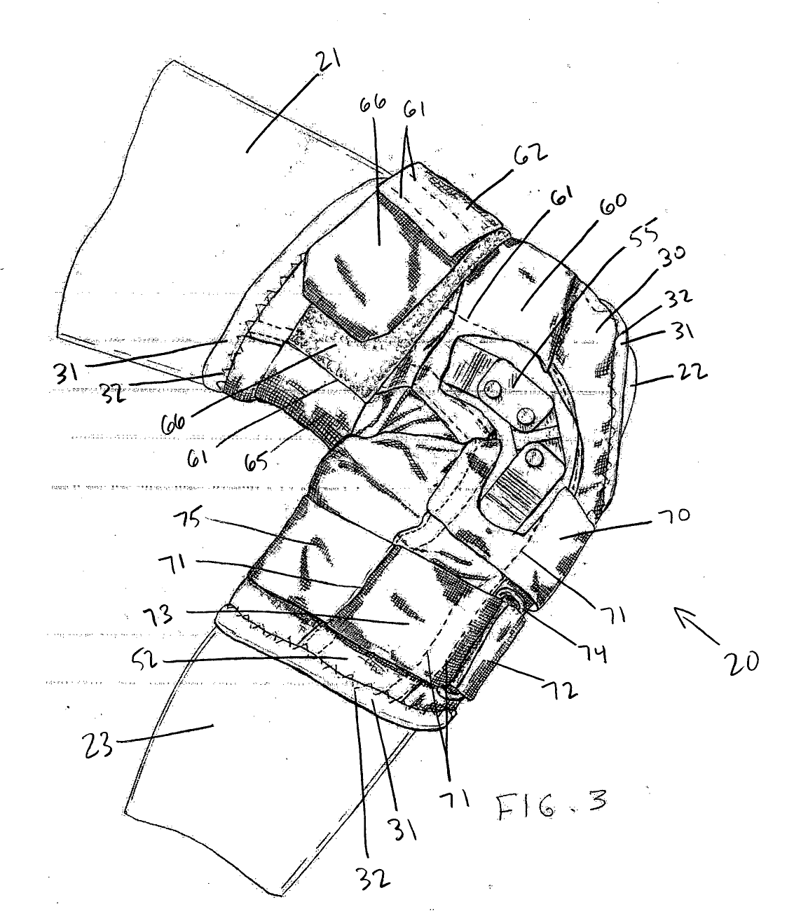

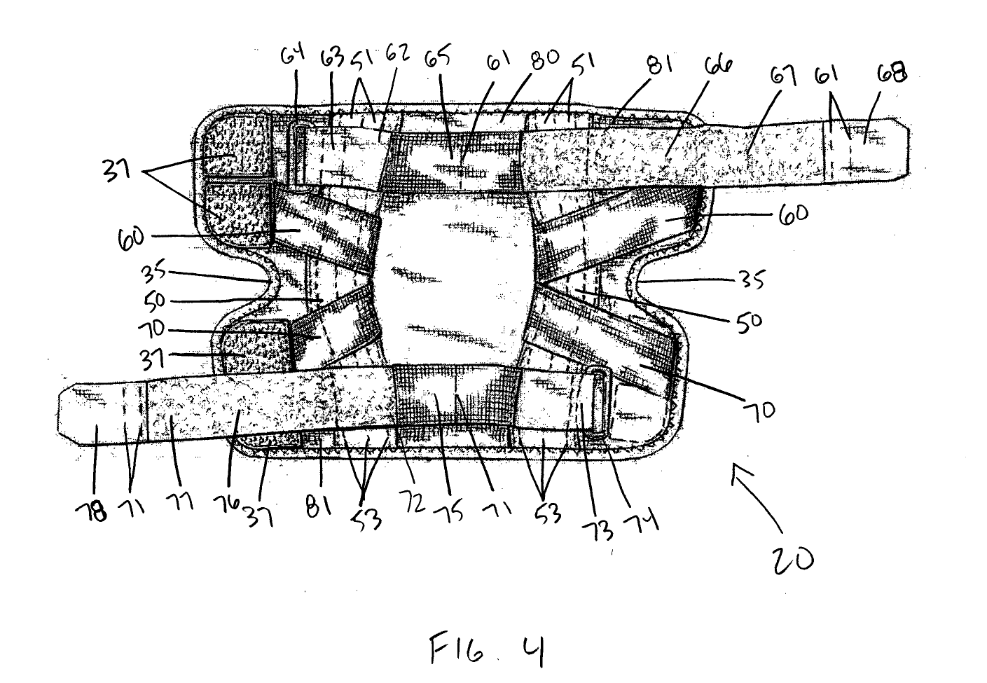

As shown in FIGS. 4-6, the base 30 is approximately rectangular in shape, with notch-shaped openings 35 on each side. As best shown in the exploded view of the base found in FIG. 6, the base 30 is formed of two side panels 81 which are permanently attached to a center panel 80, for example using stitches 82. The side panels 81 are made of elastic sheet material which stretches on the vertical direction only. The center panel 80 is made of elastic sheet material which stretches in the horizontal direction only. When the base 30 is fastened about the knee, as shown in FIGS. 1-3, the side panels 81 generally cover the front of the knee and the center panel 80 generally covers the rear of the knee. As shown in FIGS. 4 and 5, areas of hook type fastening material 37 and areas of loop type fastening material 38, of the types which adhere when pressed together, are permanently attached to the base 30, for example using stitches 39, preferably at locations corresponding roughly to the corners of the base 30.

When the base 30 is fitted upon the leg of a person, the areas of hook type fastening material 37 overlap and engage the areas of loop type fastening material 38, such that the brace can be detachably secured about the knee. As best shown in FIGS. 2 and 3, the base 30 preferably includes one or more notch-shaped openings 35 that form a hole 36 when the base 30 is fitted upon the leg of a person, whereby the kneecap 22 can extend at least partially through the hole 36.

As shown in FIGS. 1-4, one or more upper support strap segments 60 can be provided on the base 30. The ends of each upper support strap 60 are preferably permanently attached to the base 30 or an upright support assembly 50 (or both), for example using stitches 61. Lower support strap segments 70 can also be provided on the base 30. The ends of each lower support strap segment 70 are preferably permanently attached to the base 30 or an upright support assembly 50 (or both), for example using stitches 71. The upper and lower support strap segments can help provide direct support to the kneecap.

As shown in FIGS. 1-4, one or more upper fastening straps 62 and one or more lower fastening straps 72 can also be provided. As best shown in FIG. 4, an upper fastening strap 62 can include a loop segment 63, a reversing loop 64, an elastic segment 65, and a reversing segment 66. A lower fastening strap 72 can have a similar construction, including a loop segment 73, a reversing loop 74, an elastic segment 75, and a reversing segment 76.

The upper and lower fastening strap loop segments 63 and 73 are preferably formed of inelastic material and can be fixed to the base 30 or an upright support member 50 (or both), for example using stitches 61 and 71. The upper reversing loop 64 can be permanently attached to the upper loop segment 63, for example by passing an end of the upper loop segment 63 through the reversing loop 64 and fixing that end, for example using stitches 61, to the base 30 or an upright support assembly 50 (or both). Similarly, the lower reversing loop 74 can be permanently attached to the lower loop segment 73, for example by passing an end of the lower loop segment 73 through the reversing loop 74 and fixing that end, for example using stitches 71, to the base 30 or an upright support assembly 50 (or both).

The upper and lower fastening strap elastic segments 65 and 75 are preferably formed of elastic material that stretches primarily in the horizontal direction, and these fastening strap elastic segments can be fixed, for example using stitches 61 and 71, to the base 30 or an upright support assembly 50 (or both) at one or more points.

The upper and lower fastening strap reversing segments 66 and 76 are preferably formed of inelastic material and can be fixed to the base 30 or an upright support assembly 50 (or both), for example using stitches 61 and 71, respectively. The upper reversing segment 66 can include an area of loop type fastener material 67 and an area of hook type fastener material 68. Similarly, the lower reversing segment 76 can include an area of loop type fastener material 77 and an area of hook type fastener material 78.

In order to fasten the base 30 of the knee brace 20 about the leg, the base 30 can be wrapped about around the leg and the areas of hook type fastener material 37 can be pressed against the areas of loop type fastener material 38. An upper fastening strap 62 can provide additional fastening tension around the upper portion of the leg 21, for example by passing the reversing segment 66 through the reversing loop 64, pulling on the reversing segment 66 to place tension on the fastening strap 62, and pressing the area of hook material 68 against the area of loop type material 67. Similarly, a lower fastening strap 72 can provide additional fastening tension around the lower portion of the leg 23, for example by passing the reversing segment 76 through the reversing loop 74, pulling on the reversing segment 76 to place tension on the fastening strap 72, and pressing the area of hook material 78 against the area of loop type material 77.

There are various possibilities with regard to alternative embodiments and methods including a knee brace according to the invention.

Although the preferred embodiments according to the invention disclosed herein include a base formed as a reclosable sleeve, the base can also be formed as a tubular sleeve, wherein at least the portion of the tubular sleeve that forms the front of the sleeve when the sleeve is worn is made of elastic that stretches primarily in the vertical direction. The tubular sleeve can also include one or more portions, for example the portions that form the sides or rear of the base when the sleeve is worn, that are made of elastic that stretches primarily in the horizontal direction or elastic that stretches in all directions.

Although hook and loop type fastener material is preferably used to fasten the brace about the knee and to secure the upper and lower fastening straps, equivalent fasteners such as zippers, clasps, buckles, pins, laces, or buttons may be substituted for the hook and loop type fastener material.

There may be only a single upright support assembly on one side only of a knee brace according to the invention, there can be multiple upright support assemblies on one or both sides of a knee brace according to the invention. The elongated side pockets can be openable at one end to allow removal of the upright support members (the resilient stays or hinges), so that the brace may be washed or so that different upright support members may be inserted to adjust the amount and type of support provided.

Although the preferred embodiments of a knee brace according to the invention disclosed herein include upper and lower support strap segments that are permanently fixed to the base, this is not required. One or both ends of each support strap segments can be detachable, for example using hook and loop material of the type that adheres when pressed together. Although the upper and lower support strap segments are mounted in a crossing pattern, this is not required and other arrangements are possible. For example, the upper and lower fastening straps could be mounted to pass horizontally above and below the kneecap.

Although the preferred embodiments of a knee brace according to the invention disclosed herein include upper and lower fastening straps that are permanently fixed to the base, this is not required. The fastening straps need not include a reversing loop, and they need not include an elastic segment. Structures other than hook and loop material of the type that adheres when pressed together, for example clasps, buckles, pins, or buttons can also be used.

It is understood that the invention is not confined to the embodiments set forth herein as illustrative, but embraces all such forms thereof that come within the scope of the following claims.

What is claimed is:

1. A knee brace, comprising:
a base wearable in snug covering relationship to a knee and adjacent portions of the upper leg and the lower leg of a person;
the base having front, side and rear portions when the brace is worn;
wherein at least part of the front portion of the base is made of elastic material that stretches primarily in a direction from the upper leg of the person to the lower leg of the person when the brace is worn.

2. The knee brace of claim 1 further comprising one or more upright support assemblies.

3. The knee brace of claim 2 wherein each upright support assembly includes at least one elongated pocket and at least one upright support member located in the elongated pocket.

4. The knee brace of claim 3 wherein at least one upright support member includes a resilient stay.

5. The knee brace of claim 4 wherein the resilient stay is removable.

6. The knee brace of claim 4 wherein the resiliency of the resilient stay may be adjusted.

7. The knee brace of claim 3 wherein at least one upright support member includes a hinge comprising at least two links pivotally connected together and mounted to the base at a position to the side of a knee when the knee brace is in position on the leg of the person.

8. The knee brace of claim 7 wherein the upright support member is removable.

9. The knee brace of claim 1 further comprising a support strap segment having two ends, wherein at least one end of the support strap segment is fixed to the base.

10. The knee brace of claim 1 further comprising an upper support strap segment having two ends wherein at least one end of the upper support strap segment is fixed to the base and a lower support strap segment having two ends wherein at least one end of the lower support strap segment is fixed to the base.

11. The knee brace of claim 1 further comprising a support strap segment having two ends, wherein both ends of the support strap segment are fixed to the base.

12. The knee brace of claim 1 further comprising an upper support strap segment having two ends, wherein both ends of the upper support strap segment are fixed to the base and a lower support strap segment having two ends, wherein both ends of the lower support strap segment are fixed to the base.

13. The knee brace of claim 1 further comprising a fastening strap fixed to the base at one or more points.

14. The knee brace of claim 1 further comprising an upper fastening strap fixed to the base at one or more points and a lower fastening strap fixed to the base at one or more points.

15. The knee brace of claim 1 wherein the base is a tubular sleeve.

16. The knee brace of claim 1 wherein the base is an openable and reclosable sleeve.

17. A knee brace, comprising:
a base wearable in snug covering relationship to a knee and adjacent portions of the upper leg and the lower leg of a person;
the base having front, side and rear portions when the brace is worn;
wherein at least part of the front portion of the base is made of elastic material that stretches primarily in a direction from the upper leg of the person to the lower leg of the person when the brace is worn; and
wherein at least part of the rear portion of the base is made of elastic material that stretches primarily around the leg of the person when the brace is worn.

18. The knee brace of claim 17 further comprising one or more upright support assemblies.

19. The knee brace of claim 18 wherein each upright support assembly includes at least one elongated pocket and at least one upright support member located in the elongated pocket.

20. The knee brace of claim 17 further comprising a support strap segment having two ends, wherein at least one end of the support strap segment is fixed to the base.

21. The knee brace of claim 17 further comprising an upper support strap segment having two ends wherein at least one end of the upper support strap segment is fixed to the base and a lower support strap segment having two ends wherein at least one end of the lower support strap segment is fixed to the base.

22. The knee brace of claim 17 further comprising a support strap segment having two ends, wherein both ends of the support strap segment are fixed to the base.

23. The knee brace of claim 17 further comprising an upper support strap segment having two ends, wherein both ends of the upper support strap segment are fixed to the base and a lower support strap segment having two ends, wherein both ends of the lower support strap segment are fixed to the base.

24. The knee brace of claim 17 further comprising a fastening strap fixed to the base at one or more points.

25. The knee brace of claim 17 further comprising an upper fastening strap fixed to the base at one or more points and a lower fastening strap fixed to the base at one or more points.

26. The knee brace of claim 17 wherein the base is a tubular sleeve.

27. The knee brace of claim 17 wherein the base is an openable and reclosable sleeve.

28. A knee brace, comprising:
a base wearable in snug covering relationship to a knee and adjacent portions of the upper leg and the lower leg of a person;
the base having front, side and rear portions when the brace is worn; wherein at least part of the front portion of the base is made of elastic material that stretches primarily vertically when the brace is worn; and wherein at least part of the rear portion of the base is made of elastic material that stretches primarily horizontally when the brace is worn;
an upper support strap segment having two ends, wherein both ends of the upper support strap segment are fixed to the base and a lower support strap segment having two ends, wherein both ends of the lower support strap segment are fixed to the base; and
an upper fastening strap fixed to the base at one or more points and a lower fastening strap fixed to the base at one or more points.

29. The knee brace of claim 28 further comprising one or more upright support assemblies.

30. The knee brace of claim 29 wherein each upright support assembly includes at least one elongated pocket and at least one upright support member located in the elongated pocket.

31. The knee brace of claim 28 wherein the base is a tubular sleeve.

32. The knee brace of claim 28 wherein the base is an openable and reclosable sleeve.
